(19) United States
(12) Patent Application Publication
(10) Pub. No.: US 2005/0143668 A1
(43) Pub. Date: Jun. 30, 2005

(54) Automatic diagnosing method for autonomic nervous system and device thereof

(75) Inventors: Kun-Shan Lu, Taipei (TW); Cheng-Yu Wei, Hualien City (TW); Chun-Nan Teng, Kaohsiung City (TW); Terry B. J. Kuo, Chian Hsiang (TW); Ming-Sian Lin, Dongshan Township (TW)

Correspondence Address: J.C. Patents, Inc., 4 Venture, Suite 250, Irvine, CA, 92618, US

(73) Assignee: YAMAZAKI MAZAK CORPORATION, Niwa gun (JP)

(21) Appl. No.: 10/910,094

(22) Filed: Aug. 2, 2004

(30) Foreign Application Priority Data
Dec. 26, 2003 (TW) 92136982

(51) Int. Cl. A61B 5/0402

(52) U.S. Cl. 600/509

(57) ABSTRACT

An automatic diagnosing method for the autonomic nervous system and a device thereof are described. The device comprises a sensor, a computing device and an output device. An electrode of the sensor is adhered to the skin surface of a subject to detect and output the heart beat signal of the subject. The computing device collects the heart beat signal from the electrodes through the signal collection leads. The computing device further amplifies, filters, digitizes and transforms the heart beat signals into a plurality of heart rate variability parameters. Further, calculations, validation and analysis are performed on these parameters. After selecting a corresponding diagnosis description statement from a lookup table in a database, an examination report, which incorporates the diagnosis description statement and the heart rate variability parameters is output.

### CROSS-REFERENCE TO RELATED APPLICATION

This application claims the priority benefit of Taiwan application serial no. 92136982, filed on Dec. 26, 2003.

### BACKGROUNDING OF THE INVENTION

1. Field of Invention

The present invention relates to an automatic diagnosing method and an device thereof. More particularly, the present invention relates to an automatic diagnosing method for the autonomic nervous system and a device thereof, wherein a physiological signal is collected for an undisturbed period and a diagnosis description statement is outputted after the signal is analyzed.

2. Description of Related Art

The current technological advancements can provide various means for detecting and diagnosing the function of each organ in the body. However, the previous developments only focus on the accuracy of signal detection. Therefore, many invasive tools and techniques were used. For example, cardiac catheterization requires the insertion of a catheter through the artery to the heart. The procedure is not only dangerous, but also is very painful for the patient.

A non-invasive tool and technique, on the other hand, use painless and harmless approaches to detect and diagnosis the functions of the organs in the body. Since the technique and the tool are noninvasive, the accuracy of the physiological signals is usually not acceptable. Therefore, in the past, the accuracy and the practice of the noninvasive approaches are not desirable.

In recent years, Task Force of the European Society of Cardiology and the North American Society of Pacing and Electrophysiology (Heart Rate Variability: Standards of Measurement, Physiological Interpretation and Clinical Use; Circulation 93:1043-1065; 1996) and Malliani, et. al. (Cardiovascular Neural Regulation Explored in the Frequency Domain, Circulation 84:482-492; 1991) discover that besides being affected by the breathing frequency, the heart rate variability (HRV) also reflects the activity of the autonomic nervous system (ANS). An adult at rest, the heartbeat is about 60-90 beats per minute. The heart rate variability refers to the beat-to-beat alterations in the heart rate. It is a measure of the beat-to-beat, regular or irregular variations with each breath under a precordial state. Since the variation is too small, the traditional analytic methods can not provide an accurate analysis. Not only until the recent years, signal detection and treatment technique are greatly improved. Researchers have discovered that, based on frequency analysis, HRV can be characterized into two main components: the high frequency (HF) component and the low frequency (LF) component, and the low frequency component is further divided into a low frequency component and a very low frequency component. The high frequency component is synchronous with respiration and occurs every 3 seconds, whereas the exact origin of the low frequency component is not known. Investigators suspect that the low frequency component is related to vessel activity or baroreflex, and occurs every 10 seconds. Currently, physiologist and cardiologists agree that the high frequency component or the total power reflects the activity of the parasympathetic nervous system and the low frequency component is jointly contributed by both vagal and sympathetic nerves, while the ratio LF/HF is considered to mirror the activity of the sympathetic nervous system.

Besides serving as a functional indicator of the autonomic nervous system, HRV can provide meaningful reflection of many physiological conditions. For example, a recent study by Framingham further indicates that when the HRV of an elderly is lowered by one standard deviation, his/her chance of dying is about 1.7 times higher than a normal individual.

In the current non-invasive diagnosing techniques, the various parameters obtained after analysis are provided to the physician. Normally, the physician will inform the patient the result after further analyzing the parameters. However, these parameters are not meaningful to an ordinary individual who is not a medical practitioner. Therefore, the current research is focused on designing an automatic diagnosing device, wherein after the parameters are obtained and analyzed, a comprehensible description statement of the diagnosis is provided.

### SUMMARY OF THE INVENTION

Accordingly, the present invention provides an automatic diagnosing method for the autonomic nervous system (ANS), wherein a non-invasive approach is used to provide a preliminary diagnosis and recommendation on the function of the ANS. As a result, the user can obtain information regarding the activity of the autonomic nervous system and the related care.

The present invention provides an automatic diagnosing method for the autonomic nervous system, wherein an examination report and suggestion are provided for an ordinary user after the user is subjected to an easy and non-invasive examination on the activity and function of the ANS.

The present invention provides an automatic diagnosing method for the autonomic nervous system, wherein the method includes after detecting a heart beat signal of the subject, the heart beat signal is converted from the time domain to a frequency domain to obtain a plurality of the heart rate variability (HRV) parameters. A natural logarithm calculation is performed on these HRV parameters. Artificial intelligence is used to calculate and to optimize these parameters with a plurality of the corresponding reference values in the database, and a plurality of standard deviations is output. A diagnosis description statement that matches the basic information of the subject and these standard deviations is attained from a look-up table. Subsequently, an examination report, which includes the HRV parameters, the description statement of the diagnosis, the basic information and the standard deviations, is output.

In accordance to an embodiment of the present invention, the HRV parameters include the R-R intervals (peak intervals), the high frequency (HF) component, the low frequency (LF) component, and the ratio of low frequency to high frequency (LF/HF).

In accordance to the embodiment of the present invention, the above diagnosis description statement includes the standards of physiological condition, the physiological predisposition of the subject, the function of the autonomic nervous system, the age curve, the heart rate and suggestions.

In accordance to the embodiment of the present invention, the above examination report further includes the HRV parameters of the very low frequency component, the power spectrum density (PSD) and the total power.

The present invention further provides an automatic diagnosing device for the autonomic nervous system, wherein the diagnosis is achieved with a noninvasive approach. The automatic diagnosing device includes a sensing device, a computing device and an output device. The above sensing device includes a plurality of electrodes and a plurality of signal receiving leads. The electrodes are adhered to, for example, the skin surface of the subject's arm, to detect and output the heart beat signals. The computing device includes a database, wherein after receiving the heart beat signals, the signals are amplified, filtered, digitized and transformed to obtain a plurality of HRV parameters. Further, after the HRV parameters are calculated, compared and analyzed, these parameters are matched with a corresponding diagnosis description statement in the lookup table in the database. The aforementioned output device serves to receive and output an examination report, which incorporates the diagnosis description statement and the HRV parameters.

In accordance to the embodiment of the present invention, the above transformation includes fast Fourier transform.

In accordance to the embodiment of the present invention, the output device includes at least, for example, a monitor, a printer, a compact disk writer, and/or an internet system.

In accordance to the embodiment of the present invention, the above computing device includes at least an amplifier, a filter, and an analog/digital converter.

In accordance to an embodiment of the present invention, the above computing device includes a computer with a digital signal processing capability, which is used for frequency domain analysis, time domain analysis and nonlinear analysis.

In accordance to the present invention, a noninvasive automatic diagnosing device is used. The subject can be examined under a comfortable and safe environment. Further, the user can fully comprehend his/her own physiological condition even without an explanation from a medical practitioner. Moreover, the present invention can provide a preliminary diagnosis for the patient. In addition, a medical practitioner can provide a diagnosis and a treatment, which is directed to a specific part of the patient according to the examination report to rapidly and accurately eliminate the patient's illness.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic diagram illustrating an automatic diagnosing device for the autonomic nervous system according to one embodiment of the present invention.

FIG. 2 is a flow diagram illustrating a process flow of an automatic diagnosing method for the autonomic nervous system according to one embodiment of the present invention.

FIG. 3 is a flow diagram illustrating a transformation process of a heart beat signal from a time domain to a frequency domain.

FIG. 4 is a flow diagram illustrating the process of selecting a corresponding diagnosis description statement in a lookup table according to one embodiment of the present invention.

FIG. 5 is a wave diagram of electrocardiogram.

FIG. 6A is a literal part of an examination report, output from the automatic diagnosing device for the autonomic nervous system according to one embodiment of the present invention.

FIG. 6B is a graphical part of the test report, output from the automatic diagnosing device for the autonomic nervous system according to one embodiment of the present invention.

FIG. 7 is a schematic diagram illustrating the activity of the autonomic nervous system according to one embodiment of the present invention.

### DESCRIPTION OF THE PREFERRED EMBODIMENTS

The automatic diagnosing method and device thereof of the present invention is based on the “physical diagnosis technique”. The “physical diagnosis technique” refers to a method, in which instruments are used to collect blood pressure, heart rate, etc., types of physiological signals to perform medical diagnosis.

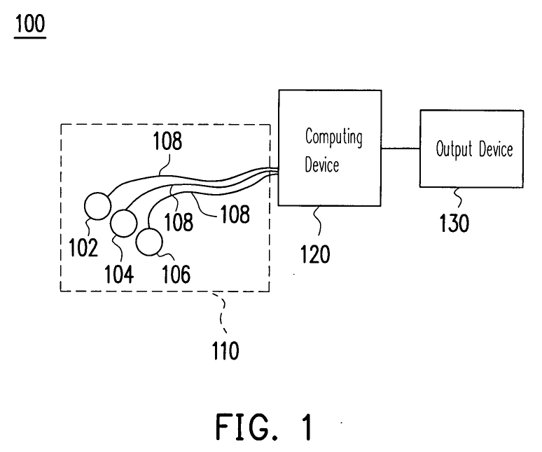

Referring to FIG. 1, FIG. 1 is a schematic diagram illustrating an automatic diagnosing device for the autonomic nervous system according to one embodiment of the present invention. In this embodiment of the present invention, the automatic diagnosing device for the autonomic nervous system employs a noninvasive approach to diagnose the function of the autonomic nervous system. The automatic diagnosing device includes a sensing device 110, a computing device 120 and an output device.

In this embodiment of the present invention, the sensing device 110 comprises a plurality of electrodes 102, 104, 106 and a plurality of signal receiving leads 108. These electrodes 102, 104, 106 are adhered to a subject, for example, to the skin surface of an arm of the subject, to detect and output the heart beat signal. A person skilled in the art can easily realize that these signal receiving leads 108 are connected to the stud electrodes, wherein one electrode is adhered to the front end of the left hand, another electrode is adhered to the back end of the left hand, while another electrode is adhered to front end of the right hand (using the standard Lead I placement), for example.

The computing device 120 includes a database (not shown), wherein this database stores a multiple of organized diagnosis description statements and an inquiry lookup table. The computing device 120 collects the heart beat signal through the signal collection leads 108. The computing device also amplifies, filters, digitizes and converts the heart beat signals to a plurality of heart rate variability (HRV) parameters. A person skilled in the art readily realizes that a computing device 120 can include, but not limited to, a plurality of high pass filters, an amplifier, a low pass filter, a voltage-current converter, a comparing circuit, an optical isolator, an analog-digital converter and a RS232 input/output port, etc.

After obtaining the HRV parameters, calculation, comparison and analysis are further performed on these parameters by the computing device. Thereafter, these parameters are matched with a corresponding diagnosis description statement in the lookup table in the database in the computing device 120.

In this embodiment, the output device 130 is coupled to the computing device 120. The output device 130 serves to receive and output the test report, which incorporates the diagnosis description statement and the heart rate variability parameters. A person skilled in the art can readily realize that the output device 130 can include a monitor, a printer to displace and print the test report, or a compact disk writer to write the test report on a compact disk. In another aspect of the invention, the examination is conducted, and the output device 130 is an internet system for sending the test report to a remote terminal, for example, the doctor's computer.

In one embodiment of the invention, the computing device 120 includes a computer with a digital signal processing (DSP) capability, which can perform frequency domain analysis, time domain analysis and nonlinear analysis.

In this embodiment of the invention, the operating principle of the automatic diagnosing device 100 used for automatically diagnosing the function of the autonomic nervous system is detailed in the following.

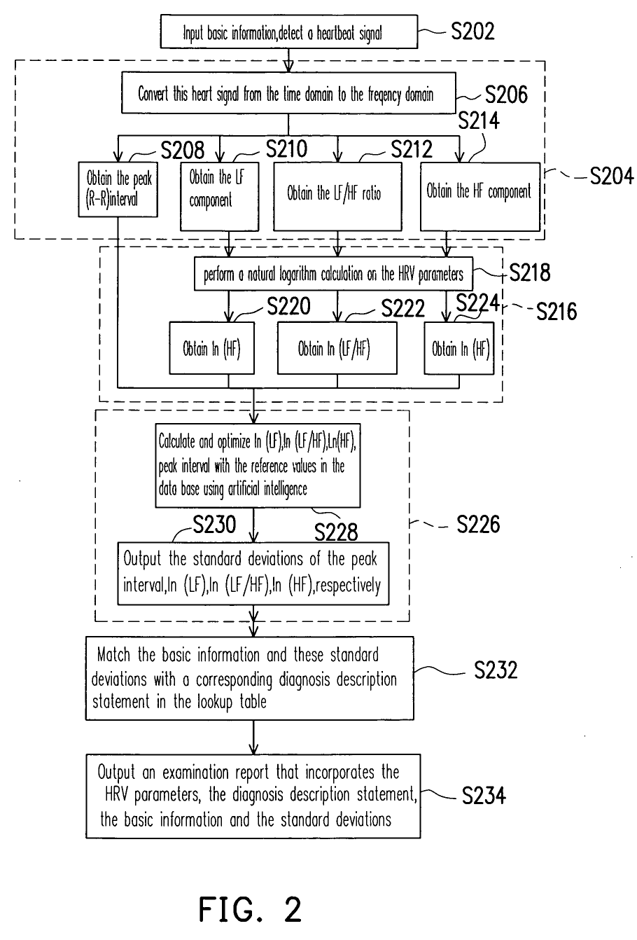

Referring to FIG. 2, FIG. 2 is a flow diagram illustrating a process flow of a method for automatically diagnosing the autonomic nervous system according to one embodiment of the present invention. This method employs a noninvasive approach to diagnosis the autonomic nervous system of a subject. In this embodiment, the heart beat signal of the subject is collected for about 5 minutes, for example.

In accordance to the automatic diagnosing method of the present invention, the basic information of the subject is first input followed by monitoring the heart beat signal (s202) of the subject. The basic information of the subject includes, but not limited to, name, age, sex, etc.

Thereafter, the heart beat signal is transformed to obtain a plurality heart rate variability (HRV) parameters (s204), wherein the step (s204) includes using fast Fourier transform to transform the heart beat signal from the time domain to the frequency domain (s206). Parameters, such as, the peak interval (the R-R interval) (s208), the low frequency (LF) component (s210), the high frequency (HF) component (s214) and the ratio of the low frequency to high frequency (s212), etc., are obtained.

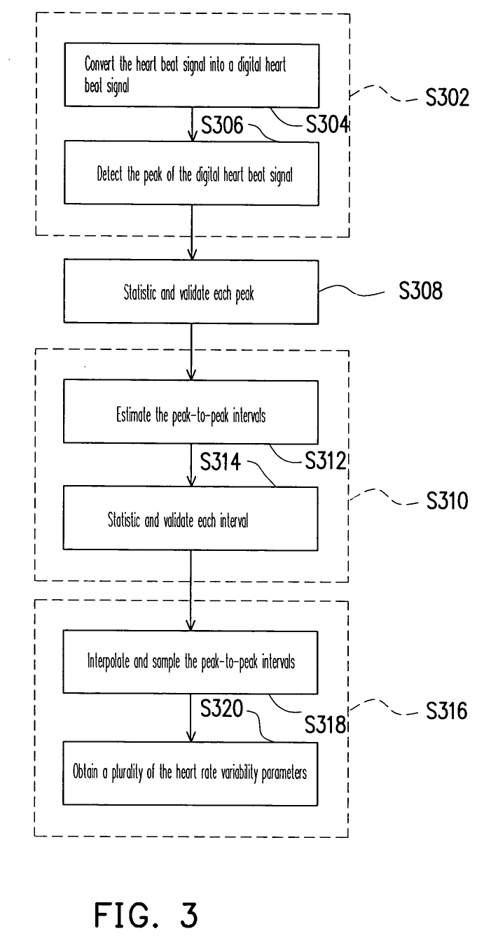

The process flow showing the transformation of the heart rate signals to obtain the plurality of HRV parameters are detailed in FIG. 3. Step (s204) in FIG. 2 includes digitally converting the heart beat signals and detecting a plurality of peaks of the heart beat signals (s302) as shown in FIG. 3.

The heart beat signals (s304) are digitally converted by using an analog/digital converter in the computing device. Thereafter, the computing device detects each peak in the digitized heart beat signals (s306).

In this embodiment, statistical validation of each peak is performed (s308) after detecting each peak. The computing device continues to calculate the peak intervals between the peaks, and statistically validate each peak interval of these peaks (s310). In fact, the computing device calculates the peak-to-peak distance to obtain a plurality of peak intervals (s312). After obtaining these peak intervals, the computing device further perform a statistical validation on each peak interval (s314).

The computing device then performs calculations on these peak intervals to obtain the heart rate variability parameters in the frequency domain (s316), wherein interpolation and sampling (s318) are performed on these peak intervals to obtain the heart rate variability parameters in the frequency domain (s320).

Referring again to FIG. 2, after obtaining these HRV parameters, in other words, after step s204, at least a natural logarithm calculation (s216) is performed on the HRV parameters using the computing device. In step (s216), the natural logarithm calculation is performed on the high frequency (HF) component and the low frequency (LF) component, and the ratio of low frequency to high frequency (LF/HF) (s218) to obtain the 1n LF (s220), the 1n (HF) (s222), and the 1n (LF/HF) (s224).

Thereafter, using a plurality of reference values in the database in the computing device, calculation and optimization are further performed on the 1n LF (s220), the 1n (HF) (s222), and the 1n (LF/HF) (s224), and a plurality of standard deviations (s266) is output. In other words, artificial intelligence is used to calculate and to optimize the peak intervals, the 1n LF (s220), the 1n (HF) (s222), and the 1n (LF/HF) (s224) with the reference values in the database in the computing device to obtain respectively the standard deviations (s230) of the peak interval, the 1n LF, the 1n (HF), and the 1n (LF/HF).

In this embodiment of the present invention, after obtaining the standard deviations of the peak intervals, the 1n (LF), the 1n (HF), and the 1n (LF/HF), respectively, a corresponding diagnosis description statement (s232) is selected from the look-up table according to the basic information of the subject and these standard deviations.

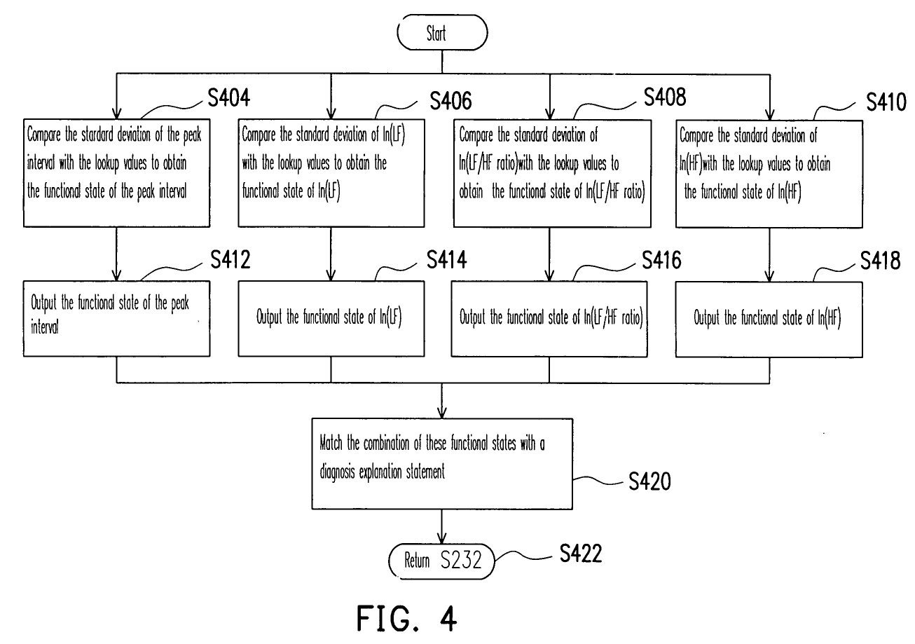

In step s232, the process flow for selecting the corresponding diagnosis description statement from the look-up table based on the basic information of the subject and the standard deviations obtained above is summarized in FIG. 4. As shown in FIG. 4, the computing device compares the standard deviations of the peak interval, the 1n (LF) component, the 1n (HF) component, the 1n (LF/HF) component with a plurality of the look-up values in the look-up table to obtain the functional state of the peak interval (s404), the functional state of 1n (LF) (s406), the function state of 1n (HF) (s410) and the functional state of 1n (LF/HF) (s408), respectively. The peak interval functional state (s404), the 1n (LF) functional state (s406), the 1n (HF) functional state (s410), the 1n (LF/HF) functional state (s408) are then output. According to the combination of these functional states, the corresponding diagnosis description statement (s420) is selected from the lookup table. Thereafter, the process flow returns to step s232 (s422).

The various functional states include the three states of low, normal and a high. Therefore, there are 3*3*3*3*3=81 combinations for the functional states of the heart rate variability parameters.

Returning to FIG. 2, in this embodiment, an examination report (s234) which incorporates the HRV parameters, the diagnosis description statement, the basic information and the standard deviations, are output (s234).

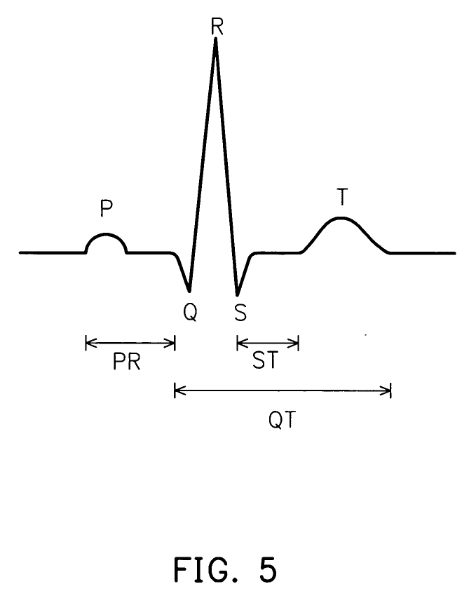

Referring to FIG. 5, FIG. 5 is a wave diagram of a heart beat signal, for example, an electrocardiograph.

In one embodiment of the present invention, the automatic diagnosing method of the autonomic nervous system detects the R pulse of the electrocardiograph from the hands of the subject for example, by transmitting the signal from the electrodes to the collection leads and further to the amplifier in the computing device for amplifying the weak signals. The QRS complex pulse (as shown in FIG. 5) is filtered out from a plurality of the noise signals and is amplified. Thereafter, an analog to digital converter is used to convert the analog signal to a digital signal.

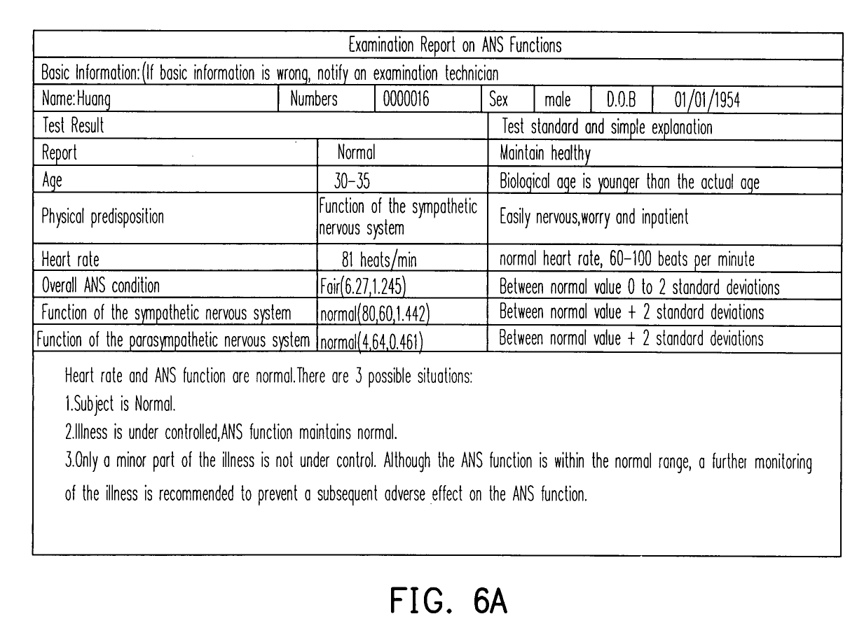

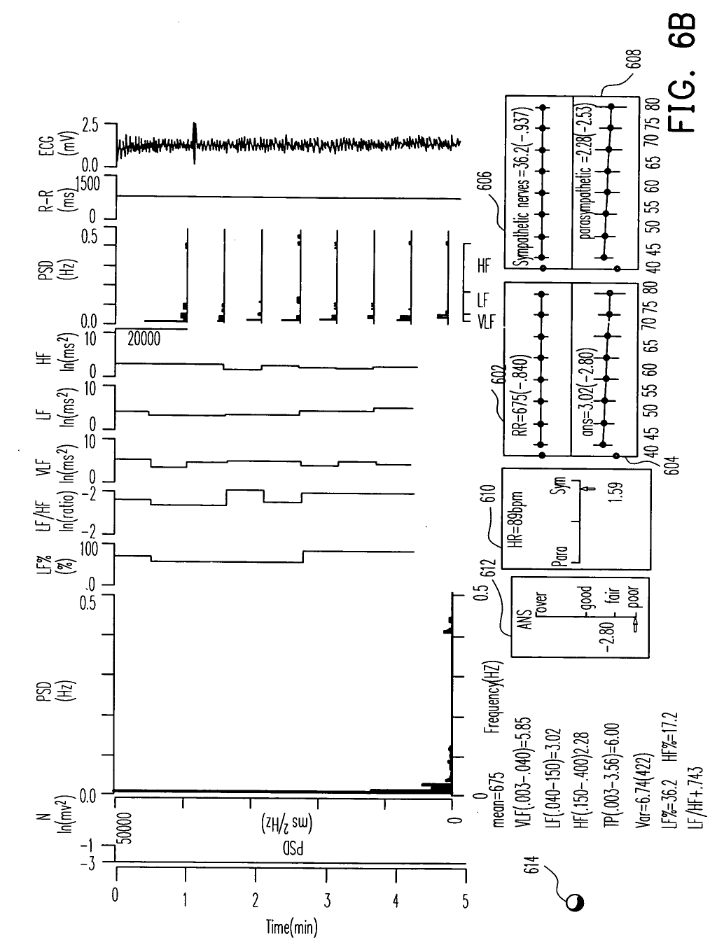

In the present embodiment, a subject will receive an examination report as shown in FIGS. 6A and 6B after being examined using the automatic diagnosing device for the autonomic nervous system of the present invention.

As shown in FIG. 6A, the literal part of the examination report includes the basic information of the subject, such as, name, identification number, date of birth and age. The examination report also includes the diagnosis description statement, which lists, for example, the results, the predisposition of the subject's physical condition, the heart rate and suggestions.

FIG. 6B illustrates the graphical part of the examination report, which includes charts, diagrams and calculated values of the various parameters for characterizing HRV.

In this embodiment, the R-R interval is a distance between two R waves in an electrocardiograph (ECG), which is also defined as the peak interval in this invention. Under a normal condition, the R-R interval is about 600 to 1000 ms. After a frequency-domain analysis is performed using the fast Fourier transform (FFT), the resulting power spectrum is quantified by means of integration into standard frequency-domain parameters including low-frequency (LF 0.04-0.15 Hz) and high-frequency (HF 0.15-0.40 Hz), total power (TP) and ratio of low frequency to high frequency (LF/HF). The high frequency component or the total power reflects the activity of the parasympathetic nervous system, the low frequency component is jointly contributed by the vagal and the sympathetic nerves, while the ratio LF/HF is considered to mirror the activity of the sympathetic nervous system. LF % represents the function and activity of the sympathetic nervous system. The power spectral density (PSD) provides the basic information of how power distributes as a function of frequency, in which at least two band powers, including the low frequency power and the high frequency power, are estimated by integrating the power spectrum. Total power (TP) is defined as a total power of all measured spectrum, which is integrated over all measured spectrum from a power density spectrum. Variance (VAR) represents the statistical variation of each value of the R-R interval during the examination period. N represents noise, in which N has to be less than −1 ln (mv²) and is normally −3 ln (mv²). If N is greater than −1 ln (mv²), the noise generated in the site must be eliminated.

In this embodiment of the invention, the curve 602 indicates an average heart beat of an individual between the age of 40 to 80. Curve 604 is an age curve, wherein the higher the black dot, the younger the subject. Curve 606 is an average activity line of the sympathetic nervous system of an individual between the age of 40 to 80, wherein a higher block dot represents the individual tends to be worrisome, excited, nervous, etc. Curve 608 is an average activity line of the parasympathetic nervous system, wherein the higher the black dot represents the individual is athletic, and the subject's sleeping pattern or the digestive system is desirable.

In this embodiment of the invention, the indicator chart 610 indicates the physiological predisposition of the subject, which can be divided into two regions. If the indicator falls between the parasympathetic nervous system and the center point, the subject tends to be less nervous or worrisome, whereas as the indicator falls between the sympathetic system and the center point, the subject tends to be more nervous or worrisome.

In this embodiment, the physical state index chart 612 indicates the physiological condition of the subject, which can be categorized into over, good, fair and poor. The position of the indicator indicates the ANS condition as recited in the diagnosis description statement.

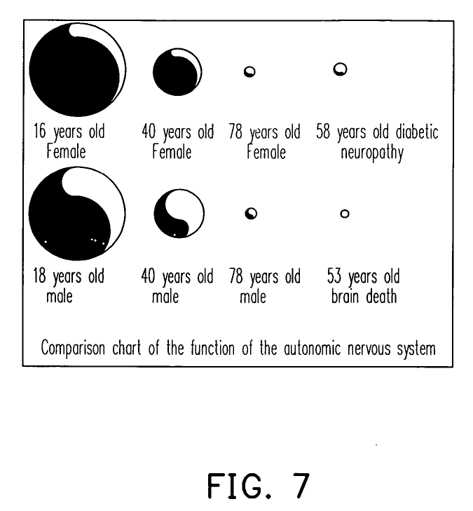

Diagram 614 illustrates the entire function activity of the autonomic nervous system, wherein the black portion represents the activity of the parasympathetic system, while the white portion represents the activity of the sympathetic system. The ratio of the black portion to the white portion is calculated based on the above HRV parameters. Referring to FIG. 7, FIG. 7 is schematic diagram illustrating the function activity of the ANS. The entire shape of the diagram serves as an indicator of the function the ANS. As shown in FIG. 7, both the age and sex affect the size and the ratio of the black portion to the white portion.

In this embodiment of the present invention, this examination report can be used to evaluate the ANS function, to predict patent outcome in the intensive care unit, to verify a brain death situation, to monitor the depth of anesthesia or heart transplant rejection, or to evaluate the aging of nervous system, etc.

In accordance to the present invention, the automatic diagnosing method for the autonomic nervous system and the device thereof, a rapid diagnosis and essential guides for diagnosing an illness can be provided. Further, an ordinary individual can also self-diagnose when abnormality of the individual's health occurs to prevent a delay in the intervention of the illness.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

1. An automatic diagnosing method, in which a non-invasive approach is used to diagnosis a function of an autonomic nervous system, the automatic diagnosing method comprising:
inputting a basic information of an user;
measuring a heart beat signal of the user;
transforming the heart beat signal into a plurality of heart rate variability (HRV) parameters;
performing at least a natural logarithm calculation on the heart rate variability parameters to obtain a plurality of natural logarithmic heart rate variability parameters;
using a plurality of reference values in a data base to calculate and optimize the natural logarithmic heart rate variability parameters and to output a plurality of standard deviations of the natural logarithmic HRV parameters;
matching the basic information and the standard deviations with a corresponding diagnosis description statement from a lookup table; and
outputting an examination report that incorporates the heart rate variability parameters, the diagnosis description statement, the basic information and the standard deviations.

2. The method of claim 1, wherein the heart rate variability parameters comprise a peak interval, a low frequency component, a high frequency component, a ratio of low frequency to high frequency and LF %.

3. The method of claim 2, wherein the step of matching the basic information and the standard deviations with the corresponding diagnosis description statement from the lookup table further comprises:
comparing the standard deviation of the peak interval with a plurality of lookup values in the lookup table to obtain a plurality of functional states of the peak interval;
comparing the standard deviation of the natural logarithmic low frequency component with the plurality of the lookup values in the lookup table to obtain a plurality of functional states of the low frequency component;
comparing the standard deviation of the natural logarithmic high frequency component with the plurality of the lookup values in the lookup table to obtain a plurality of functional states of the high frequency component;
comparing the standard deviation of the natural logarithmic ratio of low frequency to high frequency and LF % with the plurality of the lookup values in the lookup table to obtain a plurality of functional states of the ratio of low frequency to high frequency; and
outputting the corresponding diagnosis report based on the functional states of the heart rate variability parameters.

4. The method of claim 1, wherein the step transforming of the heart beat signal into the plurality of the heart rate variability parameters further comprises
digitally converting the heart beat signal and detecting a plurality of peaks of digital heart beat signal;
statistically validating each peak;
calculating a plurality of peak intervals of these peaks and statistically validating each peak interval;
performing a calculation on the peak intervals to obtain the plurality of the heart rate variability parameters.

5. The method of claim 4, wherein the step of performing the calculation on the peak intervals to obtain the plurality of the heart rate variability parameters comprises performing a fast Fourier transform.

6. The method of claim 1, wherein the examination report comprises a physical state index chart of the user, a predisposition of the user's health condition, an activity of the autonomic nervous system, an age curve, a heart rate and a suggestion.

7. The method of claim 1, wherein the examination report further comprises a very low frequency component, a total power and a power spectrum density.

8. An automatic diagnosing method for an autonomic nervous system (ANS), in which a non-invasive approach is used for diagnosing the autonomic nervous system, the method comprising
a sensing device, comprising a plurality of electrodes and a plurality of signal collection leads, wherein these electrodes are adhered to a subject to detect and output a heart rate signal of the subject;
a computing device, comprising a data base, wherein the computing device receives the heart beat signal through the signal collection leads, amplifies, filters, digitizes and transforms the heart beat signal to obtain a plurality of heart rate variability (HRV) parameters, performs a calculation and a statistical validation on the HRV parameters, and matches the HRV parameters with a corresponding diagnosis description statement from a look up table in the database; and
an output device, coupled to the computing device to receive and output an examination report that incorporates the diagnosis description statement and the HRV parameters.

9. The method of claim 8, wherein the HRV parameters comprise a peak interval, a low frequency component, a high frequency component and a ratio of low frequency to high frequency.

10. The method of claim 8, wherein the step of transforming the heart rate signals includes performing a fast Fourier transform.

11. The method of claim 8, wherein the diagnosis description statement includes a physical state index chart of the user, a predisposition of the user's health condition, an activity of the autonomic nervous system, an age curve, a heart rate and a suggestion.

12. The method of claim 8, wherein the examination report further comprises a very low frequency component, a total power and a power spectrum density.

13. The method of claim 8, wherein the output device comprises a monitor to displace the examination report.

14. The method of claim 8, wherein the output device comprises a printer to print the examination report.

15. The method of claim 8, wherein the output device comprises a compact disk writer to write the examination report on a compact disk.

16. The method of claim 8, wherein the output device further comprises a network system for sending the examination report to a remote terminal.

17. The method of claim 8, wherein the computing device comprises at least an amplifier, a filter and an analog/digital converter.

18. The method of claim 8, wherein the computing device comprises a digital signal processing capability for frequency domain analysis, time domain analysis or nonlinear analysis.

19. The method of claim 8, wherein the HRV parameters comprise a peak interval, a low frequency component, a high frequency component and LF %.
